(19) United States
(12) Patent Application Publication
(10) Pub. No.: US 2021/0346082 A1
(43) Pub. Date: Nov. 11, 2021

(54) ROBOTIC SURGICAL INSTRUMENTS AND METHODS

(71) Applicant: Covidien LP, Mansfield, MA (US)

(72) Inventors: Crystal A. Adams, Westminster, CO (US); Zachary S. Heiliger, Nederland, CO (US)

(21) Appl. No.: 17/188,771

(22) Filed: Mar. 1, 2021

Related U.S. Application Data
(60) Provisional application No. 63/021,745, filed on May 8, 2020.

(51) Int. Cl. A61B 18/14; A61B 34/30

(52) U.S. Cl. CPC A61B 18/1445; A61B 2018/00178; A61B 34/30

(57) ABSTRACT

A robotic surgical instrument includes first and second portions. The first portion includes a housing portion, a shaft including proximal, distal, and articulating segments, and first and second structural jaw bodies coupled to the distal segment of the shaft. The second portion includes an electrical cable including at least one cable body, and first and second electrically conductive plates defining tissue-contacting surfaces and electrically coupled to the cable body. The first and second portions are releasably engagable with one another in at least three locations via at least three of a first coupler coupling the cable body with the housing portion, a second coupler coupling the cable body with the proximal segment of the shaft, a third coupler coupling the cable body with the articulating portion of the shaft; or a fourth coupler coupling the first and second electrically conductive plates with the first and second structural jaw bodies, respectively.

### CROSS-REFERENCE TO RELATED APPLICATIONS

This application claims the benefit of and priority to U.S. Provisional Patent Application No. 63/021,745, filed on May 8, 2020, the entire contents of which are hereby incorporated herein by reference.

### FIELD

The present disclosure relates to surgical instruments and, more specifically, to robotic surgical instruments and methods.

### BACKGROUND

Robotic surgical systems are increasingly utilized in various different surgical procedures. Some robotic surgical systems include a console supporting one or more robotic arms. One or more different surgical instruments may be configured for use with the robotic surgical system and selectively mountable to the one or more robotic arms. The robotic arm(s) provides one or more inputs to the mounted surgical instrument(s) to enable operation of the mounted surgical instrument(s).

Surgical instruments, including robotic surgical instruments, can generally be categorized as reusable instruments (e.g., instruments that are cleaned and/or sterilized), disposable instruments (e.g., instruments that are discarded after a single use), or reposable instruments (e.g., instruments wherein portions are disposable and other portions are reusable after cleaning and/or sterilization). More recently, reprocessed instruments have been introduced by manufacturers wherein some or all components of a used instrument are refurbished, modified, and/or replaced to ready the instrument for subsequent use.

### SUMMARY

As used herein, the term “distal” refers to the portion that is being described which is farther from an operator (which may be, in robotic applications, a surgical robot), while the term “proximal” refers to the portion that is being described which is closer to the operator. The terms “about,” substantially,” and the like, as utilized herein, are meant to account for manufacturing, material, environmental, use, measurement, and other tolerances acceptable in the art. Further, to the extent consistent, any of the aspects described herein may be used in conjunction with any or all of the other aspects described herein.

Provided in accordance with aspects of the present disclosure is a robotic surgical instrument including a first portion and a second portion. The first portion includes a first portion of a housing, a shaft extending distally form the housing, and first and second structural jaw bodies. The shaft includes a proximal segment, a distal segment, and an articulating portion disposed between and interconnecting the proximal and distal segments. The first and second structural jaw bodies are coupled to the distal segment of the shaft and extend distally therefrom. At least one of the first or second structural jaw bodies is movable relative to the other and the distal segment of the shaft between a spaced-apart position and an approximated position. The second portion includes an electrical cable and first and second structural jaw bodies. The electrical cable includes at least one cable body, first and second distal electrical connectors extending distally from the at least one cable body, and an electrical plug extending proximally from the at least one cable body. The first and second structural jaw bodies define tissue-contacting surfaces and include the first and second distal electrical connectors electrically coupled thereto. The first and second portions are releasably engagable with one another in at least three locations via at least three of: a first coupler coupling the cable body with the first portion of the housing; a second coupler coupling the cable body with the proximal segment of the shaft; a third coupler coupling the cable body with the articulating portion of the shaft; or a fourth coupler coupling the first and second electrically conductive plates with the first and second structural jaw bodies, respectively.

In an aspect of the present disclosure, the first and second portions are releasably engagable with one another in at least four locations via each of the first coupler, the second coupler, the third coupler, and the fourth coupler.

In another aspect of the present disclosure, the second portion further includes a second portion of the housing having the cable body secured thereto. In such aspects, the first coupler includes complementary engagement components disposed on the first and second portions of the housing wherein engagement of the complementary engagement components couples the first and second portions of the housing with one another, thereby coupling the cable body with the first portion of the housing.

In another aspect of the present disclosure, the complementary engagement components are configured to engage one another upon approximation of the second portion of the housing relative to the first portion of the housing in at least one of: a direction generally perpendicular to a longitudinal axis of the shaft or a direction generally parallel to the longitudinal axis of the shaft.

In still another aspect of the present disclosure, the complementary engagement components include male and female snap-fit connectors or keyhole apertures and keyhole fittings.

In yet another aspect of the present disclosure, the first coupler includes a plurality of engagement features defined within and/or disposed on the first portion of the housing arranged to define a tortuous path, and the cable body is configured for receipt within the tortuous path to thereby couple the cable body with the first portion of the housing. The engagement features may include posts, recesses, protrusions, catches, undercuts, etc.

In still yet another aspect of the present disclosure, the first coupler includes a clip having the cable body secured thereto. The clip is configured to releasably engage the proximal segment of the shaft in abutment with or adjacent the first portion of the housing.

In another aspect of the present disclosure, the second coupler includes a slot and a mouth providing access to the slot. The slot and mouth are defined within the proximal segment of the shaft along at least a portion of a length thereof. The cable body is configured for insertion through the mouth and into the slot to thereby secure the cable body to the proximal segment of the shaft along the at least a portion of the length thereof.

In an aspect of the present disclosure, the second coupler includes at least one clip having the cable body attached thereto. The at least one clip is configured for engagement about the proximal segment of the shaft to thereby secure the cable body to the proximal segment of the shaft.

In yet another aspect of the present disclosure, the second coupler includes a slot and a mouth providing access to the slot. The slot and the mouth are defined within the proximal segment of the shaft. The second coupler further includes at least one clip having the cable body attached to an inwardly protruding portion thereof thereto. The at least one clip is configured for engagement about the proximal segment of the shaft. At least one of the inwardly protruding portion or cable body is configured for insertion through the mouth and into the slot to, in conjunction with the engagement of the at least one clip, thereby secure the cable body to the proximal segment of the shaft.

In still another aspect of the present disclosure, the third coupling includes securement of the cable body with the proximal and distal segments of the shaft and a slacked portion of the cable body extending through the articulating portion.

In another aspect of the present disclosure, the third coupling includes a pocket defined within at least one articulating component of the articulating portion. The cable body configured for receipt within the pocket to couple the cable body to the articulating portion of the shaft.

In still yet another aspect of the present disclosure, the fourth coupling includes first and second insulative blocks attached to the first and second electrically conductive plates, respectively, and configured for releasable engagement within the respective first and second structural jaw bodies. The engagement between the first and second insulative blocks and the first and second structural jaw bodies may be made via slot-bar engagements.

In another aspect of the present disclosure, the fourth coupling includes first and second insulative substrates attached to the first and second electrically conductive plates, respectively, each including a plurality of detents configured for releasable engagement within sockets defined within the respective first and second structural jaw bodies.

The robotic surgical instrument may further include a drive system at least partially disposed within the first housing portion. The drive system may include a jaw drive assembly for moving the at least one of the first or second structural jaw bodies between the spaced-apart position and the approximated position and/or an articulation drive assembly for articulating the distal segment of the shaft relative to the proximal segment of the shaft.

A method of assembling a robotic surgical instrument in accordance with the present disclosure includes obtaining first and second portions of a robotic surgical instrument, e.g., according to any of the aspects detailed hereinabove or otherwise herein. The method further includes coupling the first and second portions with one another via at least three of (or, in aspects, all four of): coupling the cable body with the first portion of the housing, coupling the cable body with the proximal segment of the shaft, coupling the cable body with the articulating portion of the shaft, or coupling the first and second electrically conductive plates with the first and second structural jaw bodies, respectively.

### BRIEF DESCRIPTION OF THE DRAWINGS

Various aspects and features of the present disclosure are described hereinbelow with reference to the drawings wherein like numerals designate identical or corresponding elements in each of the several views.

FIG. 1 is a perspective view of a robotic surgical instrument provided in accordance with the present disclosure;

FIG. 2 is a schematic illustration of an exemplary robotic surgical system configured to releasably receive the robotic surgical instrument of FIG. 1;

FIG. 3 is a side, simplified illustration of another robotic surgical instrument provided in accordance with the present disclosure including one or more removable components;

FIGS. 4 and 5 are exploded, top views of proximal portions of other robotic surgical instruments provided in accordance with the present disclosure including one or more removable components;

FIG. 6 is an exploded, side view of a proximal portion of still another robotic surgical instrument provided in accordance with the present disclosure including one or more removable components;

FIG. 7 is a side view of a proximal portion of yet another robotic surgical instrument provided in accordance with the present disclosure including one or more removable components;

FIGS. 8A, 8B, and 8C are top, front exploded, and front views of still yet another robotic surgical instrument provided in accordance with the present disclosure including one or more removable components;

FIGS. 9-11 are perspective views of portions of other robotic surgical instruments provided in accordance with the present disclosure including one or more removable components;

FIGS. 12 and 13 are side views of a distal portion of another robotic surgical instrument provided in accordance with the present disclosure including one or more removable components;

FIG. 14 is a transverse, cross-sectional view of a portion of the robotic surgical instrument of FIGS. 12 and 13; and

FIGS. 15 and 16 are exploded, perspective views of end effector assemblies of yet other robotic surgical instruments provided in accordance with the present disclosure including one or more removable components.

### DETAILED DESCRIPTION

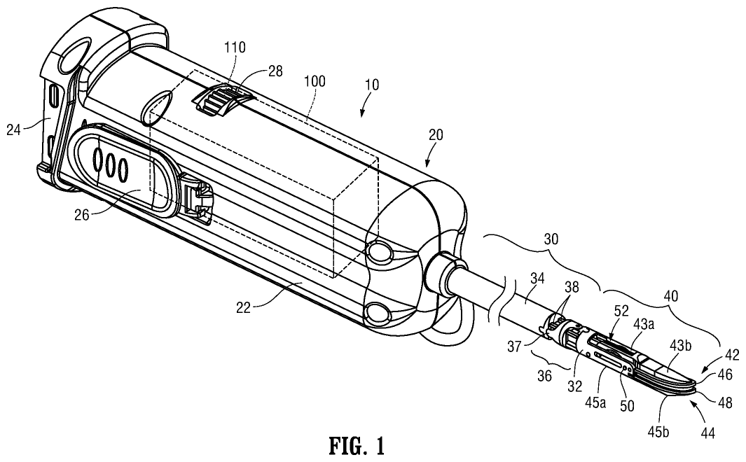

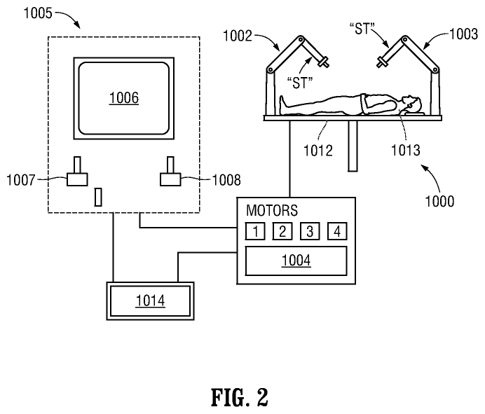

Referring to FIG. 1, a robotic surgical instrument 10 provided in accordance with the present disclosure generally includes a housing 20, a shaft 30 extending distally from housing 20, an end effector assembly 40 extending distally from shaft 30, and a drive system 100 disposed within housing 20 and operably associated with shaft 30 and end effector assembly 40. Instrument 10 is detailed herein as an articulating electrosurgical forceps configured for use with a robotic surgical system, e.g., robotic surgical system 1000 (FIG. 2). However, the aspects and features of instrument 10 provided in accordance with the present disclosure, detailed below, may also be applicable for use with other suitable surgical instruments and/or in other suitable surgical systems.

Housing 20 of instrument 10 includes a body portion 22 and a proximal face plate 24 that cooperate to define an enclosure enclosing drive system 100 therein. Proximal face plate 24 includes apertures defined therein through which inputs (not shown) of drive system 100 extend to enable operable connection of those inputs with a robotic arm, e.g., robotic arm 1002 of robotic surgical system 1000 (FIG. 2). A pair of latch levers 26 (only one of which is illustrated in FIG. 1) extends outwardly from opposing sides of body portion 22 of housing 20 and enables releasable engagement of housing 20 with the robotic arm. An aperture 28 defined through body portion 22 of housing 20 permits a thumbwheel 110 to extend therethrough to enable manual manipulation of thumbwheel 110 from the exterior of housing 20. Thumbwheel 110 is operably associated with drive system 100 such that manual manipulation of thumbwheel 110 enables manual opening and closing of end effector assembly 40.

Shaft 30 of instrument 10 includes a distal segment 32, a proximal segment 34, and an articulating section 36 disposed between the distal and proximal segments 32, 34, respectively. Articulating section 36 includes one or more articulating components 37, e.g., links, joints, etc. A plurality of articulation cables 38, e.g., four (4) articulation cables, or other suitable actuators, extends through articulating section 36. More specifically, articulation cables 38 are operably coupled to distal segment 32 of shaft 30 at the distal ends thereof and extend proximally from distal segment 32 of shaft 30, through articulating section 36 of shaft 30 and proximal segment 34 of shaft 30, and into housing 20, wherein articulation cables 38 operably couple with an articulation assembly of drive system 100 to enable selective articulation of distal segment 32 (and, thus end effector assembly 40) relative to proximal segment 34 and housing 20, e.g., about at least two axes of articulation (yaw and pitch articulation, for example). Articulation cables 38 are arranged in a generally rectangular configuration, although other suitable configurations are also contemplated.

Continuing with reference to FIG. 1, end effector assembly 40 includes first and second jaw members 42, 44, respectively. Each jaw member 42, 44 includes a proximal flange portion 43a, 45a and a distal body portion 43b, 45b, respectively. Distal body portions 43b, 45b define opposed tissue-contacting surfaces 46, 48, respectively. Proximal flange portions 43a, 45a are pivotably coupled to one another about a pivot 50 and are operably coupled to one another via a cam-slot assembly 52 including a cam pin slidably received within cam slots defined within the proximal flange portion 43a, 45a of at least one of the jaw members 42, 44, respectively, to enable pivoting of jaw member 42 relative to jaw member 44 and distal segment 32 of shaft 30 between a spaced-apart position (e.g., an open position of end effector assembly 40) and an approximated position (e.g., a closed position of end effector assembly 40) for grasping tissue between tissue-contacting surfaces 46, 48. As an alternative to this unilateral configuration, a bilateral configuration may be provided whereby both jaw members 42, 44 are pivotable relative to one another and distal segment 32 of shaft 30.

Longitudinally-extending knife channels (not shown) may be defined through tissue-contacting surfaces 46, 48, respectively, of jaw members 42, 44. In such devices, a knife assembly including a knife support (not shown) extends from housing 20 through shaft 30 to end effector assembly 40 and a knife blade (not shown) disposed within end effector assembly 40 between jaw members 42, 44 is provided to enable cutting of tissue grasped between tissue-contacting surfaces 46, 48 of jaw members 42, 44, respectively. The knife support is operably coupled to a knife drive assembly of drive system 100 at a proximal end portion thereof to enable selective actuation to reciprocate the knife blade between jaw members 42, 44 to cut tissue grasped between tissue-contacting surfaces 46, 48.

A driver (not shown) is operably coupled to cam-slot assembly 52 of end effector assembly 40, e.g., engaged with the cam pin thereof, such that actuation of the driver pivots jaw member 42 relative to jaw member 44 between the spaced-apart and approximated positions. The driver extends proximally from end effector assembly 40 through shaft 30 and into housing 20 wherein the driver is operably coupled with a jaw drive assembly of drive system 100 to enable selective actuation of end effector assembly 40 to grasp tissue therebetween.

Referring still to FIG. 1, tissue-contacting surfaces 46, 48 of jaw members 42, 44, respectively, are at least partially formed from an electrically conductive material and are energizable to different potentials to enable the conduction of electrical energy through tissue grasped therebetween, although tissue-contacting surfaces 46, 48 may alternatively be configured to supply any suitable energy, e.g., thermal, microwave, light, ultrasonic, ultrasound, etc., through tissue grasped therebetween for energy-based tissue treatment. Instrument 10 defines a conductive pathway (not shown) through housing 20 and shaft 30 to end effector assembly 40 that may include lead wires, contacts, and/or electrically conductive components to enable electrical connection of tissue-contacting surfaces 46, 48 of jaw members 42, 44, respectively, to an energy source (not shown), e.g., an electrosurgical generator, for supplying energy to tissue-contacting surfaces 46, 48 to treat, e.g., seal, tissue grasped between tissue-contacting surfaces 46, 48.

As noted above, drive system 100 is disposed within housing 20 and includes an articulation assembly, a knife drive assembly, and a jaw drive assembly. The articulation assembly may be operably coupled to first and second inputs (not shown) extending through proximal face plate 24 of housing 20 to enable coupling with corresponding outputs of the robotic arm, e.g., robotic arm 1002 of robotic surgical system 1000 (FIG. 2), such that, upon receipt of appropriate rotational inputs into the first and/or second inputs, the articulation assembly manipulates cables 38 to articulate end effector assembly 40 in a desired direction, e.g., to pitch and/or yaw end effector assembly 40. The knife drive assembly may be operably coupled to a third input (not shown) extending through proximal face plate 24 of housing 20 to enable coupling with a corresponding output of the robotic arm, e.g., robotic arm 1002 of robotic surgical system 1000 (FIG. 2), such that, upon receipt of appropriate rotational input into the third input, the knife drive assembly manipulates the knife support to reciprocate the knife blade between jaw members 42, 44 to cut tissue grasped between tissue-contacting surfaces 46, 48. The jaw drive assembly may be operably coupled to a fourth input (not shown) extending through proximal face plate 24 of housing 20 to enable coupling with a corresponding output of the robotic arm, e.g., robotic arm 1002 of robotic surgical system 1000 (FIG. 2), such that, upon receipt of appropriate rotational input into the fourth input, the jaw drive assembly pivots jaw members 42, 44 between the spaced-apart and approximated positions to grasp tissue therebetween.

Housing 20 is configured to mount on and operably interface with a robotic surgical system, e.g., system 1000 (FIG. 2), to enable robotic operation of drive system 100 to provide the above-detailed functionality. That is, robotic surgical system 1000 (FIG. 2) selectively provides rotational inputs to the four inputs of drive system 100 to articulate end effector assembly 40, grasp tissue between jaw members 42, 44, and/or cut tissue grasped between jaw members 42, 44. However, it is also contemplated that drive system 100 be configured to interface with robotic surgical system 1000 (FIG. 2) in a different manner and/or any other suitable surgical system, e.g., a manual surgical handle, a powered surgical handle, etc. For the purposes herein, robotic surgical system 1000 (FIG. 1) is generally described.

Turning to FIG. 2, robotic surgical system 1000 is configured for use in accordance with the present disclosure. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” One or more of the surgical tools “ST” may be instrument 10 (FIG. 1), thus providing such functionality on a robotic surgical system 1000.

Robot arms 1002, 1003 may be driven by electric drives, e.g., motors, connected to control device 1004. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

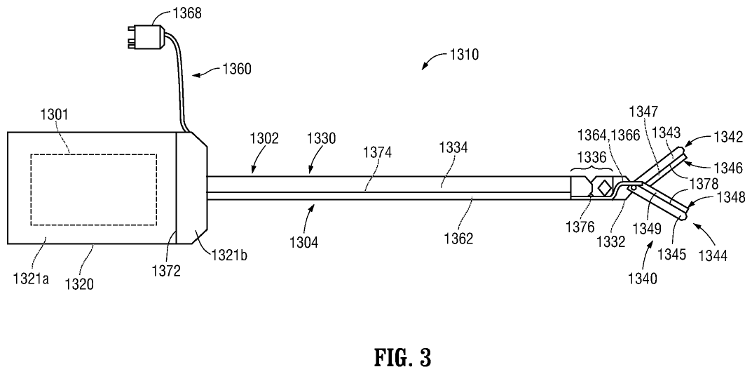

With reference to FIG. 3, another robotic surgical instrument 1310 provided in accordance with the present disclosure and configured as an articulating electrosurgical forceps configured for use with a robotic surgical system, e.g., robotic surgical system 1000 (FIG. 2), is shown. To the extent consistent and unless specifically contradicted below, robotic surgical instrument 1310 may include any of the aspects and features of robotic surgical instrument 10 (FIG. 1) detailed above. Accordingly, only differences therebetween are described in detail hereinbelow while similarities are omitted or summarily described.

Instrument 1310 generally includes a housing 1320, a shaft 1330 extending distally from housing 1320, an end effector assembly 1340 extending distally from shaft 1330, and a drive system 1301 disposed within housing 1320 and operably associated with shaft 1330 and end effector assembly 1340. Shaft 1330 includes a distal segment 1332, a proximal segment 1334, and an articulating section 1336 disposed between the distal and proximal segments 1332, 1334. End effector assembly 1340 includes first and second jaw members 1342, 1344, respectively, each having a structural body 1343, 1345, e.g., including a proximal flange portion and distal body portion, and opposed tissue-contacting surface 1346, 1348 disposed on the distal body portion of the corresponding structural body 1343, 1345. Structural bodies 1343, 1345 pivotably couple jaw members 1342, 1344 to one another and distal segment 1332 of shaft 1330. One of both of structural bodies 1343, 1345 is also operably coupled with the driver to enable pivoting of jaw member 1342 and/or jaw member 1344 between the spaced-apart and approximated positions for grasping tissue between opposed tissue-contacting surface 1346, 1348.

An electrical cable 1360 of instrument 1310 houses lead wires or other electrically conductive components and includes a cable body 1362, distal electrical connectors 1364, 1366, and a proximal electrical plug 1368. Distal electrical connectors 1364, 1366 are electrically coupled to tissue-contacting surfaces 1346, 1348 of jaw members 1342, 1344, respectively, while proximal electrical plug 1368 is configured to connect to an energy source (not shown), e.g., an electrosurgical generator, thus enabling the supply of energy to tissue-contacting surfaces 1346, 1348 to treat, e.g., seal, tissue grasped between tissue-contacting surfaces 1346, 1348. Cable body 1362 extends between and electrically couples distal electrical connectors 1364, 1366 with proximal electrical plug 1368. In some aspects, rather than housing both lead wires or other electrically conductive components in a single cable body 1362, electrical cable 1360 includes two separate cable bodies 1362, each carrying the lead wire or other electrically conductive component for one of the tissue-contacting surfaces 1346, 1348 of jaw members 1342, 1344, respectively. In such configurations, the couplings detailed below may be duplicated as necessary to enable coupling of both cable bodies 1362, e.g., to shaft 1330. Additional cable bodies 1362, distal electrical connectors 1364, 1366, and/or electrical plugs 1368 may be provided for additional functionality, e.g. a cutting electrode of end effector assembly 1340, a sensor associated with end effector assembly 1340, etc.

Instrument 1310 may be configured as a single-use instrument that is discarded after use or sent to a manufacturer for reprocessing; a reusable instrument capable of being cleaned and/or sterilized for repeated use by the end-user; or a partially-single-use, partially-reusable instrument. With respect to partially-single-use, partially-reusable configurations, some portions of instrument 1310 may be configured as cleanable/sterilizable, reusable components, while other components are configured as single-use, disposable/reprocessable components. Whether for the purpose of facilitating cleaning/sterilizing of all components, cleaning/sterilizing of some components and replacement of others, or for other purposes, instrument 1310 is capable of being disassembled into at least first and second portions 1302, 1304, respectively, and re-assembled with the same or replacement portions (or components thereof).

First portion 1302, in aspects, may include a portion 1321a or the entirety of housing 1320 (with portion 1321a at least partially housing drive system 1301), shaft 1330, structural bodies 1343, 1345 of jaw members 1342, 1344, respectively, and the internal operable components extending through housing 1320 and/or shaft 1330, e.g., drive system 1301, the driver (not shown), the knife support and knife blade (not shown), and the articulation cables (not shown). First portion 1302 may be configured as a cleanable/sterilizable reusable portion (with or without some components of first portion 1302 being replaced between uses).

Second portion 1304, in aspects, may include a portion 1321b of housing 1320, electrically conductive tissue-contacting surfaces 1346, 1348 (including, for example, electrically conductive plates 1347, 1349 defining the respective electrically conductive tissue-contacting surfaces 1346, 1348) of jaw members 1342, 1344, respectively, and electrical cable 1360. In some configurations, second portion 1304 further includes insulators and/or other support or attachment structures associated therewith, as detailed below. Second portion 1304 may be configured as a single-use, disposable portion. Alternatively, first and second portions 1302, 1304 may be releasably engagable to enable different types of components to be used, e.g., to enable user-selection of different jaw features, different electrical plugs configurations for different generators, etc., and/or to facilitate cleaning/sterilization for reuse, manufacture, packaging, shipping, storage, etc.

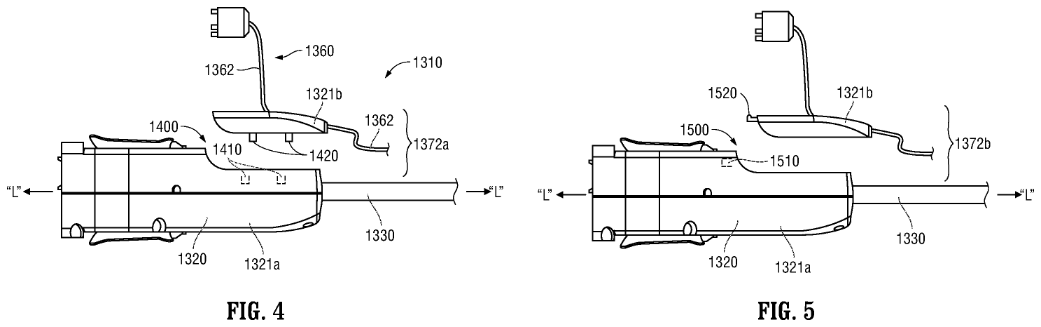

Continuing with reference to FIG. 3, in order to maintain proper positioning of first and second portions 1302, 1304, respectively, relative to one another for use while also facilitating disassembly and reassembly of first and second portions 1302, 1304, respectively, from one another, various coupling features are provided for releasably coupling first and second portions 1302, 1304 with one another at various locations along instrument 1310. For example: a first coupling 1372 may be provided to couple portions 1321a, 1321b of housing 1320 with one another; a second coupling 1374 may be provided to couple cable body 1362 of electrical cable 1360 with proximal segment 1334 of shaft 1330; a third coupling 1376 may be provided to couple cable body 1362 of electrical cable 1360 with articulating section 1336 of shaft 1330; and/or a fourth coupling 1378 may be provided to couple electrically conductive plates 1347, 1349 of jaw members 1342, 1344 with respective structural bodies 1343, 1345 of jaw members 1342, 1344. Several configurations for each of these couplings 1372, 1374, 1376, 1378 are detailed below with reference to FIGS. 4-16, and can be used in any suitable combination. Further, in some configurations all four couplings 1372, 1374, 1376, 1378 are utilized; in other configurations only three of the couplings 1372, 1374, 1376, 1378 are utilized; in still other configurations only two of the couplings 1372, 1374, 1376, 1378 are utilized; and in yet other configurations, only one of the couplings 1372, 1374, 1376, 1378 is utilized. Other additional or alternative couplings are also contemplated.

Referring to FIG. 4, a configuration of first coupling 1372a is shown wherein portions 1321a, 1321b of housing 1320 are configured to releasably couple to one another via a snap-fit engagement. Portion 1321b of housing 1320 includes cable body 1362 of electrical cable 1360 secured thereto, routed therethrough, or otherwise coupled therewith such that the coupling of portions 1321a, 1321b of housing 1320 with one another retains at least a portion of cable body 1362 in position relative to housing 1320. Portion 1321a of housing 1320 may define a recess 1400 configured to receive portion 1321b of housing 1320 such that, upon coupling of portions 1321a, 1321b of housing 1320 with one another a substantially uninterrupted outer surface of housing 1320 is defined. Other configurations are also contemplated.

Continuing with reference to FIG. 4, portions 1321a, 1321b of housing 1320 each include one or more snap-fit connector portions 1410, 1420, e.g., male engagement tab(s) or female engagement opening(s). Snap-fit connector portions 1410 may all be the same as one another, e.g., female engagement opening(s), with snap-fit connector portions 1420 being the same as one another and complementary to snap-fit connector portions 1410, e.g., male engagement tabs, although the reverse configuration or mix configurations wherein snap-fit connector portions 1410, 1420 each include one or more female engagement openings and one or more male engagement tabs, are also contemplated. In aspects, male engagement tabs of snap-fit connector portions 1410, 1420 are configured to break, deform, or otherwise prevent re-engagement with female engagement openings of snap-fit connector portions 1410, 1420 after an initial engagement and disengagement. Such a configuration inhibits re-engagement of first coupling 1372a and, thus, inhibits reuse of first portion 1302 and/or second portion 1304. As an alternative to snap-fit connectors, other suitable connectors are also contemplated such as, for example, tape, magnets, latches, etc.

In order to engage portions 1321a, 1321b of housing 1320 with one another, portion 1321b is approximated relative to recess 1400 of portion 1321a in generally perpendicular orientation relative to a longitudinal axis “L” defined by housing 1320 and shaft 1330 until portion 1321b is received at least partially within recess 1400 of portion 1321a and snap-fit connector portions 1410, 1420 engage one another, e.g., as confirmed by audible and/or tactile “snaps.” Disengagement of portions 1321a, 1321b is achieved in the opposite manner, e.g., moving portions 1321a, 1321b apart from one another in generally perpendicular orientation relative to the longitudinal axis “L.”

Recess 1400 defined within portion 1321a of housing 1320 may communicate with the interior of portion 1321a that houses at least a portion of drive system 1301 therein, or a floor separating recess 1400 from the interior of portion 1321a and drive system 1301 may be provided. That is, portion 1321b of housing 1320 may define a functional portion of the enclosure housing drive system 1301 therein, or may attach to an exterior of portion 1321a with portion 1321a defining the enclosure housing drive system 1301. In either configuration, with portions 1321a, 1321b engaged with one another, a portion of cable body 1362 (the portion attached to portion 1321b of housing 1320) is retained in position at housing 1320 to avoid interference with the manipulation and/or use of instrument 1310.

FIG. 5 illustrates another configuration of first coupling 1372b. First coupling 1372b is similar to first coupling 1372a (FIG. 4) and may include any of the features thereof. First coupling 1372b differs from first coupling 1372a (FIG. 4) in that snap-fit connector portions 1510, 1520 are positioned such that portions 1321a, 1321b of housing 1320 are engagable with one another via approximating portion 1321b relative to recess 1500 of portion 1321a in generally parallel orientation relative to the longitudinal axis “L” defined by housing 1320 and shaft 1330 until portion 1321b is received at least partially within recess 1500 and snap-fit connector portions 1510, 1520 engage one another, e.g., as confirmed by audible and/or tactile “snaps.” Disengagement is accomplished in the opposite manner.

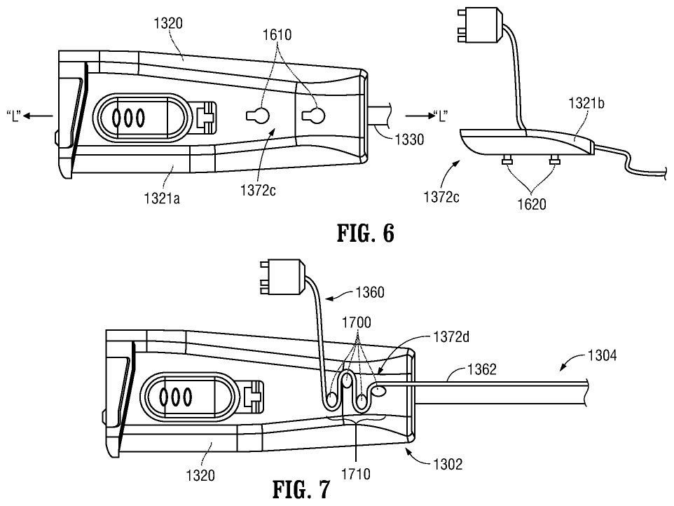

With reference to FIG. 6, still another configuration of first coupling 1372c is shown wherein portion 1321a defines one or more keyhole apertures 1610 and/or key fittings 1620 and wherein portion 1321b defines one or more corresponding keyhole apertures 1610 and/or keyhole fittings 1620. Although not shown, the keyhole apertures 1610 and/or keyhole fittings of portion 1321a may be disposed at least partially within a recess configured to receive second portion 1321b.

In order to engage portions 1321a, 1321b of housing 1320 with one another via first coupling 1372c, portion 1321b is first approximated relative to portion 1321a in generally perpendicular orientation relative to the longitudinal axis “L” defined by housing 1320 and shaft 1330 until the keyhole fittings 1620 are received within the relatively large portions of the corresponding keyhole apertures 1610. Thereafter, portion 1321b is approximated relative to portion 1321a in generally parallel orientation relative to the longitudinal axis “L” such that the keyhole fittings 1620 are slid into the relatively small portions of the corresponding keyhole apertures 1610, thereby engaging portions 1321a, 1321b with one another. Disengagement is accomplished in the opposite manner.

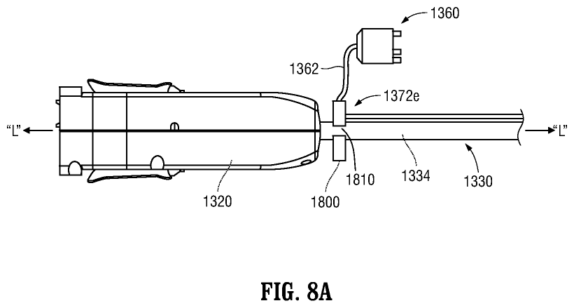

As illustrated in FIG. 7, yet another configuration of first coupling 1372d is provided. With respect to first coupling 1372d, the entirety of housing 1320 is part of first portion 1302 and, thus, no part of housing 1320 constitutes second portion 1304. Coupling 1372d instead includes a plurality of engagement features, e.g., posts 1700, extending from housing 1320 and arranged to define a tortuous path 1710, e.g., a path including a plurality of bends, through the posts 1700. In order to couple electrical cable 1360 with housing 1320, a portion of cable body 1362 is routed through the tortuous path 1710 defined by posts 1700 to thereby retain that portion of cable body 1362 in position relative to housing 1320. Disengagement is accomplished via removing cable body 1362 from tortuous path 1710. Posts 1700 may be linear, as shown, or may define any other suitable configurations such as, for example, T-shaped, L-shaped, angled, curved, etc. Further, as an alternative or in addition to posts 1700, other suitable engagement features defined within and/or disposed on first portion 1302 of housing 1320 may be provided such as, for example, recesses, channels, tunnels, protrusions, catches, undercuts, clips, etc.

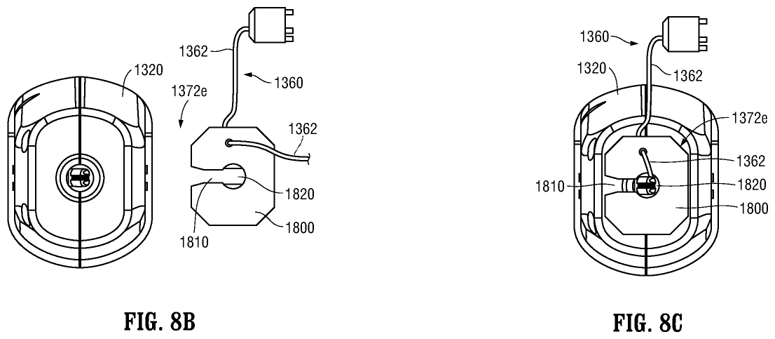

FIGS. 8A-8C show still yet another configuration of first coupling 1372e. As with first coupling 1372d (FIG. 7), first coupling 1372e is utilized where the entirety of housing 1320 is part of first portion 1302 and, thus, no part of housing 1320 constitutes second portion 1304. Coupling 1372e instead includes a clip 1800 defining a mouth 1810 and a throat 1820 and is configured to clip onto proximal segment 1334 of shaft 1330. Clip 1800 may define a U-shaped configuration, a C-shaped configuration, or any other suitable configuration enabling transverse insertion about and engagement with shaft 1330, as detailed below. Clip 1800 includes cable body 1362 of electrical cable 1360 secured thereto, routed therethrough, or otherwise coupled therewith to retain that portion of cable body 1362 in position relative to clip 1800. Clip 1800 is configured for positioning on shaft 1330 in abutment with or adjacent to housing 1320, e.g., within about 10 cm thereof, to thereby indirectly couple cable body 1362 with housing 1320.

In order to engage clip 1800 with proximal segment 1334 of shaft 1330, clip 1800, led by mouth 1810, is approximated relative to proximal segment 1334 of shaft 1330 transversely relative thereto, e.g., generally perpendicularly relative to the longitudinal axis “L” defined by housing 1320 and shaft 1330, until proximal segment 1334 of shaft 1330 is at least partially received within mouth 1610. Mouth 1610 defines a diameter equal to or smaller than proximal segment 1334 of shaft 1330 such that, upon further transverse urging of clip 1800 relative to shaft 1330, mouth 1610 is expanded to enable proximal segment 1334 of shaft 1330 to enter throat 1820. With proximal segment 1334 of shaft 1330 received within throat 1820, mouth 1810 is retuned towards its initial position to retain clip 1800 in fixed position and engagement about proximal segment 1334 of shaft 1330 with proximal segment 1334 of shaft 1330 extending through throat 1820. Disengagement is accomplished in the opposite manner.

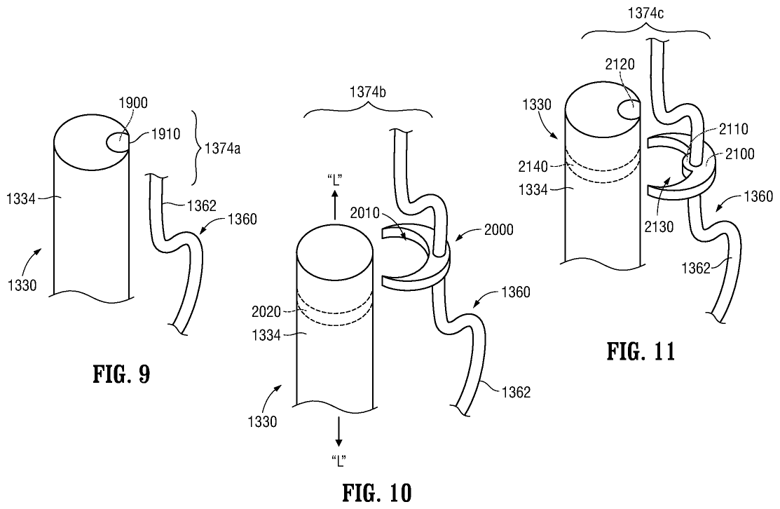

Referring to FIGS. 9-11, various configurations of the second coupling 1374 (FIG. 3) are illustrated. As noted above, the second coupling 1374 (FIG. 3) may be provided to couple cable body 1362 of electrical cable 1360 with proximal segment 1334 of shaft 1330. With reference to FIG. 9, a second coupling 1374a is shown wherein proximal segment 1334 of shaft 1330 defines a slot 1900 extending along at least a portion of a length thereof. In aspects, slot 1900 extends at least 50%, at least 70%, or at least 90% of the length of proximal segment 1334 of shaft 1330. Slot 1900 defines a generally circular cross-sectional configuration. A mouth 1910 extending along the length of slot 1900 is disposed in communication with slot 1900. Slot 1900 defines an internal diameter that generally approximates, e.g., within 15%, the outer diameter of cable body 1362 of electrical cable 1360, while mouth 1910 defines an opening that is equal to or slightly smaller, e.g., within 15%, than the outer diameter of cable body 1362 of electrical cable 1360. Proximal segment 1334 of shaft 1330 may be composite.

In order to engage cable body 1362 within slot 1900, cable body 1362 is urged transversely through mouth 1910, resiliently compressing cable body 1362 and/or resiliently expanding mouth 1910 to enable passage of cable body 1362 through mouth 1910 and into slot 1900. With cable body 1362 engaged within slot 1900 as detailed above, cable body 1362 does not protrude radially outwardly beyond the outer circumference of proximal segment 1334 of shaft 1330 and, thus, does enlarge the outer diameter of proximal segment 1334 of shaft 1330. Due to the close-fit engagement of cable body 1362 within slot 1900, cable body 1362 is retained in fixed position relative to proximal portion 1334 of shaft 1330 substantially along the length of proximal portion 1334 of shaft 1330.

As shown in FIG. 10, another second coupling 1374b is provided including a clip 2000 having cable body 1362 secured thereto, routed therethrough, or otherwise coupled therewith. Cable body 1362, more specifically, is coupled to clip 2000 in a manner that does not interfere with or extend into the interior of clip 2000. Clip 2000 defines a C-shaped configuration with an internal diameter that generally approximates, e.g., within 15%, the outer diameter of proximal segment 1334 of shaft 1330, and a mouth 2010 that defines an opening slightly smaller, e.g., within 15%, than the outer diameter of proximal segment 1334 of shaft 1330. In embodiments, plural clips 2000 are provided for engagement about proximal segment 1334 of shaft 1330 at spaced-apart positions along at least a portion of the length thereof. As an alternative to one or more separate clips 2000, elongated clips (not shown), e.g., formed via extrusion, may be provided to similarly engage proximal segment 1334 of shaft 1330 along a more substantial portion or substantially all of the length thereof as compared to clips 2000. The elongated clips may have a similar transverse cross-sectional configuration as any of the other clips detailed herein and may be formed from a flexible material, e.g., rubber, or any other suitable material to facilitate engagement about proximal segment 1334 of shaft 1330 along at least a portion of the length thereof.

In order to engage clip 2000 about proximal segment 1334 of shaft 1330, clip 2000, led by mouth 2010, is approximated relative to proximal segment 1334 of shaft 1330 transversely relative thereto, e.g., generally perpendicularly relative to the longitudinal axis “L” of shaft 1330, until proximal segment 1334 of shaft 1330 passes through mouth 2010 (expanding clip 2000) and into clip 2000 (whereby clip 2000 resiliently returns). Disengagement is accomplished in the opposite manner. In some configurations, one or more annular or partially-annular recesses 2020 may be defined within proximal segment 1334 of shaft 1330 for receipt of clip(s) 2000 therein such that clip(s) 2000 protrudes less or does not protrude radially outwardly beyond the outer circumference of proximal segment 1334 of shaft 1330. Additionally or alternatively, a slot 1900 (FIG. 9) as detailed above with respect to second coupling 1374a (FIG. 9) may be utilized in conjunction with annular recesses 2020 such that cable body 1362 likewise protrudes less or does not protrude radially outwardly beyond the outer circumference of proximal segment 1334 of shaft 1330. Complementary anti-rotation features (not shown), e.g., protrusions and recesses, pins and slots, angled surfaces, etc., disposed on and/or defined within clip 2000 and annular recesses 2020 may be provided to rotationally lock clip 2000 relative to proximal segment 1334 of shaft 1330.

With reference to FIG. 11, another second coupling 1374c includes a clip 2100 having cable body 1362 secured thereto, routed therethrough, or otherwise coupled therewith. In embodiments, plural clips 2100 are provided for engagement about proximal segment 1334 of shaft 1330 at spaced-apart positions along at least a portion of the length thereof. Cable body 1362, more specifically, is coupled to an inwardly protruding portion 2110 of clip 2100 that extends into the interior of clip 2100. Further, a slot 2120 similar to slot 1900 (FIG. 9) is defined along proximal segment 1334 of shaft 1330. Slot 2120 is configured to receive, through mouth 2130 of clip 2100, cable body 1362 similarly as detailed above with respect to second coupling 1374a (FIG. 9) and is further configured to receive inwardly protruding portion 2110 of clip 2100. Clip 2100 is configured for engagement about proximal segment 1334 of shaft 1330 similarly as detailed above with respect to clip 2000 (FIG. 10). In some configurations, one or more annular recesses 2140 is defined within proximal segment 1334 of shaft 1330 for receipt of clip(s) 2100 therein such that clip(s) 2100 protrudes less or does not protrude radially outwardly beyond the outer circumference of proximal segment 1334 of shaft 1330. Slot 2120 transversely intersects annular recesses 2140 such that, while the body of clip 2100 is received within annular recesses 2140, inwardly protruding portion 2110 of clip 2100 and cable body 2162 are received within slot 2120.

Generally referring to FIGS. 9-11, in conjunction with FIG. 3, it is noted that, in some configurations, any of the above-detailed second couplings 1374a, 1374b, 1374c may likewise be utilized to couple cable body 2162 with distal segment 1332 (FIG. 3) of shaft 1330, similarly as detailed above with respect to proximal segment 1334 of shaft 1330.

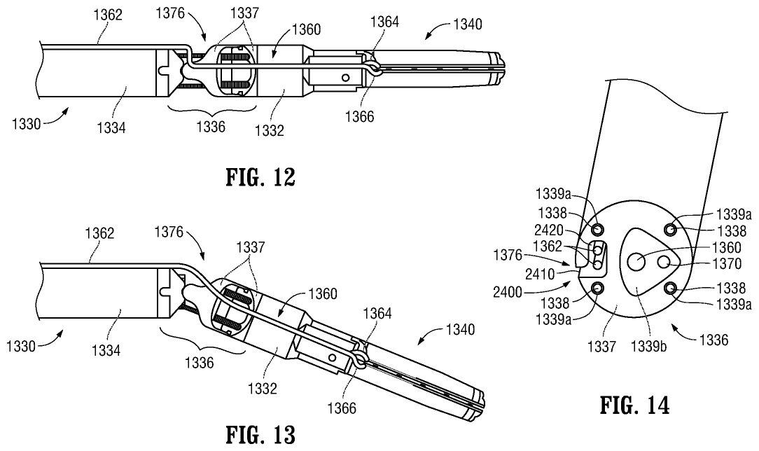

FIGS. 12 and 13 illustrate a third coupling 1376 wherein cable body 1362 of electrical cable 1360 is secured to proximal segment 1334 of shaft 1330, e.g., as detailed above via engagement within a slot and/or through the use of clips or in any other suitable manner; is routed distally through articulating section 1336 of shaft 1330; and is secured to distal segment 1332 of shaft 1330, e.g., as detailed above via engagement within a slot and/or through the use of clips or in any other suitable manner. In some configurations, distal segment 1332 is omitted and/or cable body 1362 is not secured to distal segment 1332 but is routed distally through articulating section 1336 whereafter distal electrical connectors 1364, 1366 couple to end effector assembly 1340. Cable body 1362, more specifically, is routed distally through articulating section 1336 of shaft 1330 with sufficient slack to enable articulation of articulating section 1336 without inhibiting the range of motion thereof.

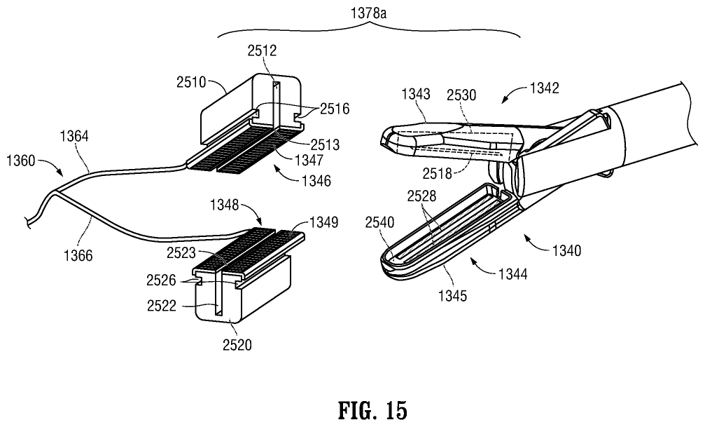

Cable body 1362 may extend alongside articulating section 1336 of shaft 1330 free of direct coupling thereto, thus allowing slack to be taken up or let out as shaft 1330 articulates, or may be coupled to or within articulating section 1336 at one or more locations that enable sliding of cable body 1362 therethrough to allow slack to be taken up or let out as shaft 1330 articulates. With reference to FIG. 14, for example, one or more of the articulating component(s) 1337 of articulating section 1336 may include a pocket 2400 defined therethrough and extending longitudinally therealong. Each pocket 2400 defines an entry opening 2410 and an internal cavity 2420 communicating with the entry opening 2410. Opening 2410 and cavity 2420 may be disposed at an angle relative to one another, e.g., of from about 60 degrees to about 120 degrees, to enable insertion of cable body 1362 through opening 2410 and into cavity 2420 while inhibiting cable body 1362 from being dislodged from pocket 2400. As shown in FIG. 14 and as noted above, in some configurations, two (or more) cable bodies 1362 may be provided; alternatively, a single cable body 1362 may be provided.

Pocket 2400 is configured to retain the one or more cable bodies 1362 therein as noted above; however, cavity 2420 defines sufficiently large dimensions relative to the one or more cable bodies 1362 to enable longitudinal, e.g., proximal and distal, sliding of cable bodies 1362 within and relative to cavity 2420 and the articulating component 1337 defining cavity 2420 to enable slack to be taken up or let out of cable bodies 1362 as shaft 1330 articulates. This is in contrast to first and second couplers 1372, 1374 (FIG. 3) which, in at least some configurations, are configured to substantially inhibit movement of portions of cable body 1362 relative to housing 1320 and/or shaft 1330. In other configurations sliding of cable body 1362 relative to the first and second couplers 1372, 1374 (FIG. 3) is permitted.

Continuing with reference to FIG. 14, in addition to pocket 2400 defined within articulation component 1337, articulation component 1337 further defines articulation cable lumens 1339a receiving articulation cables 1338 therethrough, and an actuator lumen 1339b receiving the driver 2460 and knife bar 2470 therethrough. Lumens 1339a, 1339b are separate from pocket 2400, although in some aspects, lumen 1339b may communicate with pocket 2400.

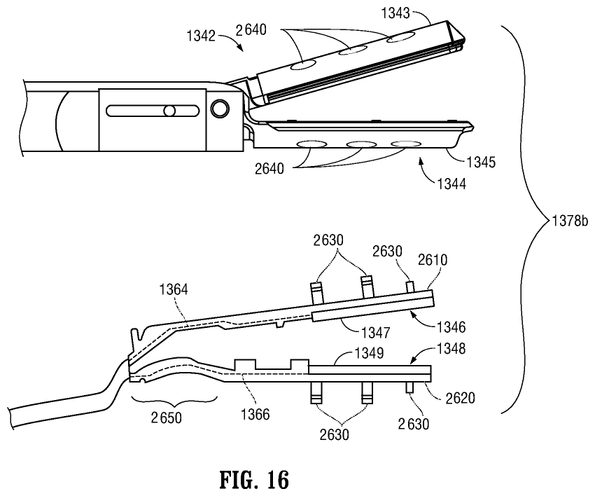

Referring to FIG. 15, a fourth coupling 1378a is illustrated to couple electrically conductive plates 1347, 1349 of jaw members 1342, 1344 with respective structural bodies 1343, 1345 of jaw members 1342, 1344, respectively. Distal electrical connectors 1364, 1366 of electrical cable 1360 (FIG. 3) are electrically connected to electrically conductive plates 1347, 1349, respectively. Electrically conductive plates 1347, 1349 are disposed on, e.g., adhered to, secured to via overmolding, or otherwise attached to, insulator blocks 2510, 2520. Insulator blocks 2510, 2520 serve to electrically isolate plates 1347, 1349 from structural bodies 1343, 1345, respectively, when engaged therewith. One or both of the insulator blocks 2510, 2520 define knife channels 2512, 2514 that align with the knife channel 2513, 2515 of the corresponding electrically conductive plate 1347, 1349 such that, when end effector assembly 1340 is assembled and in use, translation of a knife blade (not shown) through the knife channels may be effected to cut tissue grasped between jaw members 1342, 1344.

Insulator blocks 2510, 2520 are configured for receipt within cavities 2530, 2540, respectively, defined within respective structural bodies 1343, 1345. Blocks 2510, 2520 and cavities 2530, 2540 include complementary releasable engagement features, e.g., male and female components, interlocking components, etc., to facilitate releasable engagement of insulator blocks 2510, 5520 within respective structural bodies 1343, 1345. For example, as illustrated in FIG. 15, insulator blocks 2510, 2520 may define longitudinal slots 2516, 2526 extending along opposed sides thereof that are configured for receipt within corresponding longitudinal bars 2518, 2528 extending inwardly from structural bodies 1343, 1345 into cavities 2530, 2540 on opposing sides thereof to enable releasable engagement of insulator blocks 2510, 2520 within respective structural bodies 1343, 1345. Via this engagement, electrically conductive plates 1347, 1349 are disposed on respective structural bodies 1343, 1345 and positioned such that tissue-contacting surfaces 1346, 1348 oppose one another.

With reference to FIG. 16, another fourth coupling 1378b is illustrated to couple electrically conductive plates 1347, 1349 of jaw members 1342, 1344 with respective structural bodies 1343, 1345 of jaw members 1342, 1344, respectively. With respect to fourth coupling 1378b, electrically conductive plates 1347, 1349 are disposed on, e.g., adhered to, secured to via overmolding, or otherwise attached to, insulative substrates 2610, 2620 that serve to electrically isolate plates 1347, 1349 from structural bodies 1343, 1345, respectively, when engaged therewith. Each substrate 2610, 2620 includes a plurality of bifurcated detents 2630 configured to compress during insertion through sockets 2640 defined within structural bodies 1343, 1345 and to expand to releasably engage structural bodies 1343, 1345 upon sufficient insertion into sockets 2640 to thereby releasably engage insulative substrates 2610, 2620 (having electrically conductive plates 1347, 1349 disposed thereon) with structural bodies 1343, 1345. Distal electrical connectors 1364, 1366 of electrical cable 1360 (FIG. 3) are electrically connected to electrically conductive plates 1347, 1349, respectively, by way of a flexible, insulative interconnect 2650. With substrates 2610, 2620 engaged with respective structural bodies 1343, 1345, electrically conductive plates 1347, 1349 are positioned such that tissue-contacting surfaces 1346, 1348 oppose one another.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A robotic surgical instrument, comprising:
a first portion, including:
a first portion of a housing;
a shaft extending distally from the first portion of the housing, the shaft including a proximal segment, a distal segment, and an articulating portion disposed between and interconnecting the proximal and distal segments; and
first and second structural jaw bodies coupled to the distal segment of the shaft and extending distally therefrom, at least one of the first or second structural jaw bodies movable relative to the other and the distal segment of the shaft between a spaced-apart position and an approximated position; and
a second portion, including:
an electrical cable including at least one cable body, first and second distal electrical connectors extending distally from the at least one cable body, and an electrical plug extending proximally from the at least one cable body; and
first and second electrically conductive plates defining tissue-contacting surfaces, wherein the first and second distal electrical connectors are electrically coupled to the first and second electrically conductive plates, respectively,
wherein the first and second portions are releasably engagable with one another in at least three locations via at least three of:
a first coupler coupling the cable body with the first portion of the housing;
a second coupler coupling the cable body with the proximal segment of the shaft;
a third coupler coupling the cable body with the articulating portion of the shaft; or
a fourth coupler coupling the first and second electrically conductive plates with the first and second structural jaw bodies, respectively.

2. The robotic surgical instrument according to claim 1, wherein the first and second portions are releasably engagable with one another in at least four locations via each of the first coupler, the second coupler, the third coupler, and the fourth coupler.

3. The robotic surgical instrument according to claim 1, wherein the second portion further includes a second portion of the housing having the cable body secured thereto, and wherein the first coupler includes complementary engagement components disposed on the first and second portions of the housing, wherein engagement of the complementary engagement components couples the first and second portions of the housing with one another, thereby coupling the cable body with the first portion of the housing.

4. The robotic surgical instrument according to claim 3, wherein the complementary engagement components are configured to engage one another upon approximation of the second portion of the housing relative to the first portion of the housing in at least one of: a direction generally perpendicular to a longitudinal axis of the shaft or a direction generally parallel to the longitudinal axis of the shaft.

5. The robotic surgical instrument according to claim 3, wherein the complementary engagement components include male and female snap-fit connectors or keyhole apertures and keyhole fittings.

6. The robotic surgical instrument according to claim 1, wherein the first coupler includes a plurality of engagement features at least one of defined within or disposed on the first portion of the housing arranged to define a tortuous path, and wherein the cable body is configured for receipt within the tortuous path to thereby couple the cable body with the first portion of the housing.

7. The robotic surgical instrument according to claim 1, wherein the first coupler includes a clip having the cable body secured thereto, the clip configured to releasably engage the proximal segment of the shaft in abutment with or adjacent the first portion of the housing.

8. The robotic surgical instrument according to claim 1, wherein the second coupler includes a slot and a mouth providing access to the slot, the slot and mouth defined within the proximal segment of the shaft along at least a portion of a length thereof, the cable body configured for insertion through the mouth and into the slot to thereby secure the cable body to the proximal segment of the shaft along the at least a portion of the length thereof.

9. The robotic surgical instrument according to claim 1, wherein the second coupler includes at least one clip having the cable body attached thereto, the at least one clip configured for engagement about the proximal segment of the shaft to thereby secure the cable body to the proximal segment of the shaft.

10. The robotic surgical instrument according to claim 1, wherein the second coupler includes a slot and a mouth providing access to the slot, the slot and mouth defined within the proximal segment of the shaft along at least a portion of a length thereof, the second coupler further including at least one clip having the cable body attached to an inwardly protruding portion thereof thereto, wherein the at least one clip configured for engagement about the proximal segment of the shaft, and at least one of the inwardly protruding portion or cable body configured for insertion through the mouth and into the slot to thereby secure the cable body to the proximal segment of the shaft.

11. The robotic surgical instrument according to claim 1, wherein the third coupling includes securement of the cable body with the proximal and distal segments of the shaft and a slacked portion of the cable body extending through the articulating portion.

12. The robotic surgical instrument according to claim 1, wherein the third coupling includes a pocket defined within at least one articulating component of the articulating portion, the cable body configured for receipt within the pocket to couple the cable body to the articulating portion of the shaft.

13. The robotic surgical instrument according to claim 1, wherein the fourth coupling includes first and second insulative blocks attached to the first and second electrically conductive plates, respectively, and configured for releasable engagement within the respective first and second structural jaw bodies.

14. The robotic surgical instrument according to claim 13, wherein the first and second insulative blocks are configured for releasable engagement within the respective first and second structural jaw bodies via slot-bar engagements.

15. The robotic surgical instrument according to claim 1, wherein the fourth coupling includes first and second insulative substrates attached to the first and second electrically conductive plates, respectively, each including a plurality of detents configured for releasable engagement within sockets defined within the respective first and second structural jaw bodies.

16. The robotic surgical instrument according to claim 1, wherein the first portion further includes a drive system at least partially disposed within the first housing portion, the drive system including:
a jaw drive assembly operably coupled to the at least one of the first or second structural jaw bodies via a driver such that actuation of the jaw drive assembly moves the drive to thereby move the at least one of the first or second structural jaw bodies between the spaced-apart position and the approximated position; and
an articulation drive assembly operably coupled to the articulation portion of the shaft via a plurality of articulation cables such that actuation of the articulation drive assembly moves at least some of the plurality of articulation cables to thereby articulate the distal segment of the shaft relative to the proximal segment of the shaft.

17. A method of assembling a robotic surgical instrument, comprising:
obtaining a first portion of a robotic surgical instrument, the first portion including:
a first portion of a housing;
a shaft extending distally from the first portion of the housing, the shaft including a proximal segment, a distal segment, and an articulating portion disposed between and interconnecting the proximal and distal segments; and
first and second structural jaw bodies coupled to the distal segment of the shaft and extending distally therefrom, at least one of the first or second structural jaw bodies movable relative to the other and the distal segment of the shaft between a spaced-apart position and an approximated position;
obtaining a second portion of the robotic surgical instrument, the second portion including:
an electrical cable including at least one cable body, first and second distal electrical connectors extending distally from the at least one cable body, and an electrical plug extending proximally from the at least one cable body; and
first and second electrically conductive plates defining tissue-contacting surfaces, wherein the first and second distal electrical connectors are electrically coupled to the first and second electrically conductive plates, respectively; and
coupling the first and second portions with one another via at least three of:
coupling the cable body with the first portion of the housing;
coupling the cable body with the proximal segment of the shaft;
coupling the cable body with the articulating portion of the shaft; or
coupling the first and second electrically conductive plates with the first and second structural jaw bodies, respectively.

18. The method according to claim 17, wherein coupling the first and second portions with one another includes coupling each of:
the cable body with the first portion of the housing;
the cable body with the proximal segment of the shaft;
the cable body with the articulating portion of the shaft; or
the first and second electrically conductive plates with the first and second structural jaw bodies, respectively.
